(19) United States
(12) Patent Application Publication
(10) Pub. No.: US 2020/0065553 A1
(43) Pub. Date: Feb. 27, 2020

(54) Remote sensing architecture utilizing multiple UAVs to construct a sparse sampling measurement matrix for a compressed sensing system

(71) Applicants: BUJIN GUO, Rosenberg, TX (US); XIAODAN LI, Orlando, FL (US); JAMES M. GUO, Rosenberg, TX (US)

(72) Inventors: BUJIN GUO, Rosenberg, TX (US); XIAODAN LI, Orlando, FL (US); JAMES M. GUO, Rosenberg, TX (US)

(21) Appl. No.: 16/112,729

(22) Filed: Aug. 26, 2018

(51) Int. Cl. G06K 9/00; B64C 39/02; G05D 1/00

(52) U.S. Cl. CPC G06K 9/0063; B64C 39/024; G05D 1/0027; G06K 2009/00644; B64C 2201/127; B64C 2201/123; B64C 2201/146

(57) ABSTRACT

The present disclosure relates to a remote sensing system which comprises at least a plurality of UAVs or drones to construct a measurement matrix in space for compressed sensing algorithms. A single remote sensor or a plural remote sensors is carried by each of UAV or drone in the fleet and can be independently turned on or turned off. Each UAV or drone in the fleet works as a floating pixel in said sparse measurement matrix, to output sampling data for processing and reconstruction of sensing image by said compressed sensing algorithm.

### CROSS REFERENCE TO RELATED APPLICATIONS

This application is in-part a continuation of the U.S. provisional Patent Application Ser. No. 62/551,208, filed Aug. 28, 2017, and entitled “Remote sensing architecture utilizing UAVs as sparse sampling matrix for compressed sensing system”.

### FIELD OF THE INVENTION

The present invention relates generally to remote sensing, more particularly, to a remote sensing architecture that utilizes a fleet of drones, or UAVs, each of which carries one or more sensors, to create sparse sampling measurement matrices for a compressed sensing system.

### BACKGROUND OF THE INVENTION

Remote sensing is most effective for detecting substances in a large area and is used in numerous fields such as defense, natural resource exploration, agriculture and environmental protection.

In remote sensing systems, various types of sensors are used for a variety of applications in the area of detecting, ranging, mapping, topography and geological survey. Chemical sensors used to detect chemical substances, gases such as carbon dioxide and methane. Physical sensors detect physical parameters such as distance, speed, acceleration, pressure, density and temperature. Optical sensors detect optical properties, such as reflection, interference, imaging, and spectral characteristics.

In the area of remote sensing, the development of hyperspectral imaging has emerged as a very promising technology. Hyperspectral cameras deployed in airborne or satellite systems can cover the visible and near-infrared wavelengths to provide a wide array of spectral information for numerous applications. Different acquisition techniques for hyperspectral imaging allow data to be visualized as sections of the hyperspectral data cube with its two spatial dimensions (x, y) and one sensor dimension (lambda). There are four basic techniques for acquiring this multi-dimensional (x, y, and lambda) dataset of the data cube: spatial scanning, spectral scanning, non-scanning and spatial-spectral scanning. Various remote sensing methods and apparatuses are used to detect physical, chemical, thermal, spectral, and biological properties of the objects as stated above. There are two basic scanner designs for remote sensing. A “whisk-broom” scanner uses an oscillating mirror to scan terrain reflectance along scan lines perpendicular to the sensor's flight line. The second design is a “push-broom” scanner that uses a linear array of sensor elements to simultaneously record reflectance at uniform intervals along the scan line. No matter what acquisition technique is employed and what property of the target is to be explored, due to extremely large volume of data received during processing, compression in data acquisition has become a critical factor in obtaining satisfactory measurements.

Compressed sensing is a signal processing technique for efficiently acquiring and reconstructing a signal. For convenience of illustration, the abbreviation “CS” will be used to identify “Compressed sensing” in the description hereafter. This is based on the principle that, through optimization, the sparsity of a signal can be exploited to recover it from far fewer samples than required by the Shannon-Nyquist sampling theorem. Theoretically, there are two conditions under which recovery is possible. The first one is sparsity which requires the signal to be sparse in some domain. The second is incoherence which is applied through the isometric property which is sufficient for sparse signals. CS has attracted considerable attention and now has been successfully used in various applications. In the area of remote sensing, CS is one of the most common data compression approaches to exploiting signal's sparsity to substantially reduce the data file size and to obtain faster overall results.

One of the most notable and widely recognized achievements of CS is a single-pixel camera designed by Rice University, which is a new type of camera architecture based on a digital micro mirror device (DMD) and a single detector element. The DMD includes a two-dimensional array of programmable micro mirrors, each of which is configured to independently and controllably switch between two orientation states. With help of DMD, the camera can scan an image with much fewer measurements than a pixel-by-pixel scanning system to drastically reduce data size, saving processing time and yielding system performance.

In recent years, autonomous drones, a type of Unmanned Aerial Vehicle (UAV), have been developed with explosive growth and have captured the global market attention. The amazing versatility of drones, achieved by its multiple flight modes and patterns, and affordability, achieved by its dramatic price drop trend in recent and upcoming years, has attracted many developers to explore potential drone applications. The number of emerging applications of drones has increased in a handful of industries including aerial surveillance, agriculture, land management, energy, utilities, mining and others. People have reason to believe that, in the future, commercial drones will open up even more possibilities in applications.

The primary objective of this invention is to provide a unique remote sensing architecture utilizing multiple UAVs, such as a fleet of drones, equipped with a single or plural of sensors to construct a CS measurement matrix in 3-D space.

In compressed sensing system, measurement matrix plays a very significant role in signal sampling and signal reconstruction. Recently, much research has been conducted on design of new measurement matrix, because the implementation of CS largely depends on utilizing a suitable random or structured measurement matrix. Measurement matrix design is not the focus in this discussion, and current invention is not limited to work only for one type of CS algorithm.

### BRIEF SUMMARY OF THE INVENTION

The present invention presents a novel remote sensing architecture utilizing UAVs such as a fleet of drones. The fleet of drones or UAVs is patterned in the space to form a sparse measurement matrix based on the modulation sequence of a compressed sensing algorithm, to provide sampling data for CS processing and reconstruction. Each drone in the CS measurement matrix is equipped with one or a plurality of airborne sensors and functions as a floating pixel in the sparse matrix, to output sampling data for CS processing and reconstruction. The said sensors can be programed on or off independently according to CS algorithms.

### BRIEF DESCRIPTION OF THE DRAWINGS

Some preferred embodiments of the present invention are illustrated by accompanying figures, but the current invention is not limited to the embodiments set forth herein. For purposes of exemplification, drone and fleet of drones are used in present embodiments but are not limited to. Any UAV or other type of aerial vehicle can be used as sensor carrier shown further in the embodiments of FIGS.

FIG. 1 and FIG. 2 are explanatory views of current invention. FIG. 1 shows drone 100, which is one of the drones in drone fleet 200 shown in FIG. 2, equipped with multiple sensors 101.

FIG. 2 shows a fleet of drones 200 forming a single or plural sparse measurement matrices in 3-D space. The drone matrix pattern is designed and controlled by CS algorithms for data sampling and collecting.

FIG. 3 illustrates a prior art CS system for a single pixel camera.

FIG. 4 is a schematic block diagram to illustrate an embodiment of the current invention, to use a fleet of drones as measurement matrix for CS data processing and image reconstruction.

FIG. 5 shows a drone swarm 500, which will be rearranged as shown in FIG. 7 to create a sparse measurement matrix for CS system.

FIG. 6 shows a sampling pattern of measurement matrix 600 designed by a CS algorithm.

FIG. 7 illustrates one embodiment of current invention. The pattern of measurement matrix 600, required by CS algorithm in FIG. 6, is embodied by utilizing a fleet of drones. The measurement matrix 700, corresponding to pattern 600, is accomplished by a fleet of drones composed of multiple identical drones 701.

FIG. 8 is a schematic block diagram to demonstrate another embodiment of the current invention, by utilizing an array of drones, as shown in FIG. 9, to construct CS measurement matrix.

FIG. 9 shows an array of drones 900, composed of multiple identical drone 901. Sensors at each drone in drone array 900 can be independently turned on or turned off.

FIG. 10 shows an embodiment of current invention described in FIG. 8. The pattern of measurement matrix 600, required by CS algorithm in FIG. 6, is embodied by utilizing drone array 900 shown in FIG. 9. The CS measurement matrix 1000, corresponding to pattern 600, is accomplished by independently turning on or off sensors at each drone in array 900, such as turning on sensors at drone 1001, turning off sensors at drone 1002, and so on.

FIG. 11 shows one embodiment of drones working in CS measurement matrix. Drone 1100 in CS matrix is equipped with multiple sensors, sensor 1101, sensor 1102, sensor 1103 and more, which can be any type of sensors or sensor combinations, such as physical sensors (temperature sensors, pressure sensors, magnetic field sensors, radiation detectors, etc.), chemical sensors (various gas detectors, pH sensors, etc.), photonics sensors (imaging sensors of all spectral band, etc.), and so on.

FIG. 12 shows another embodiment of drones working in CS measurement matrix. Drone 1200 in CS matrix is equipped with a miniature spectrometer 1201 for hyperspectral imaging measurement.

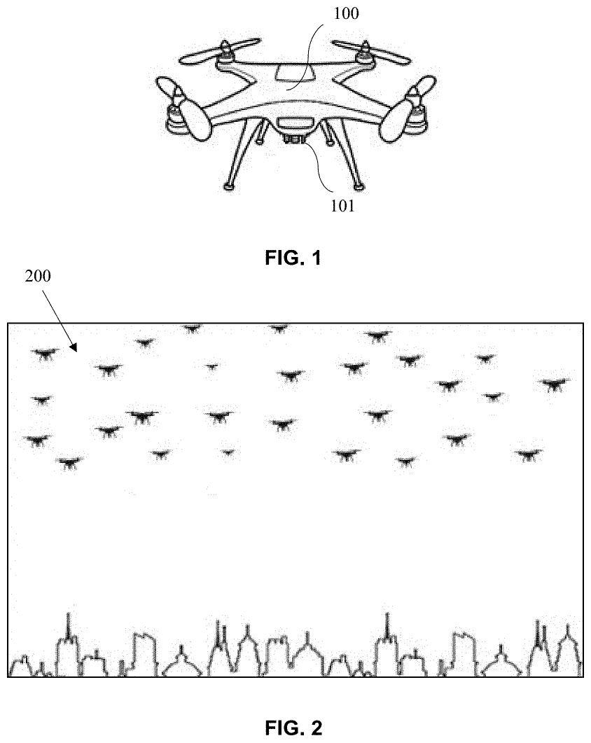

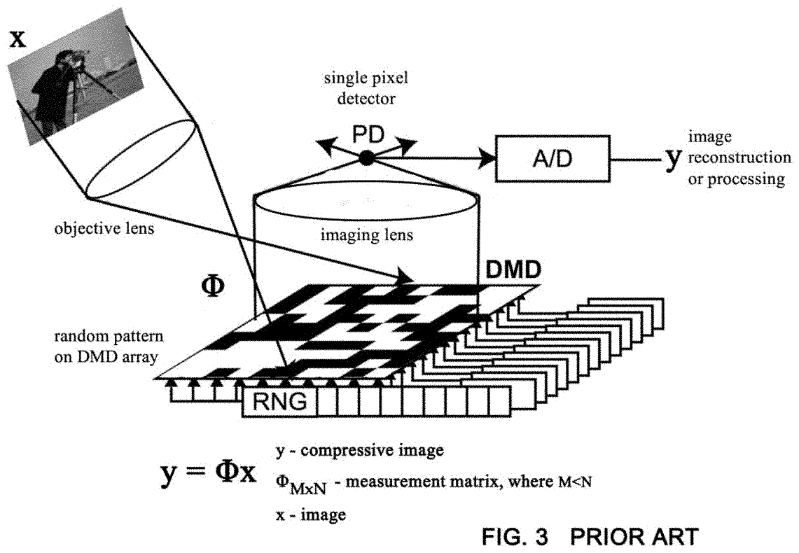

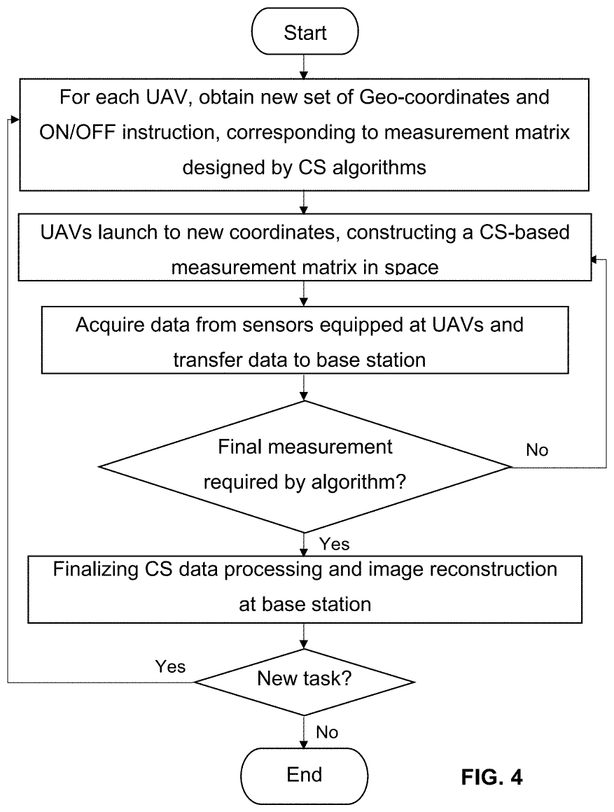

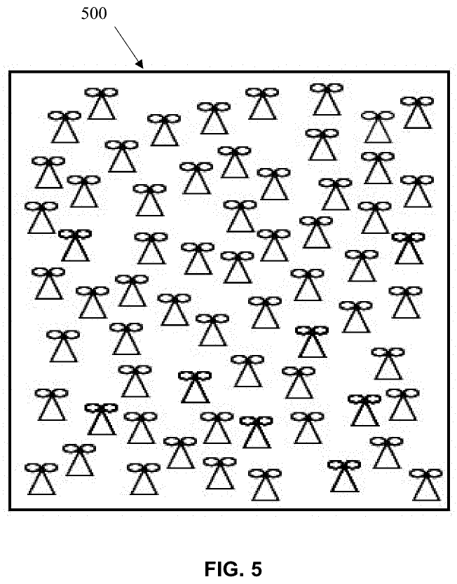

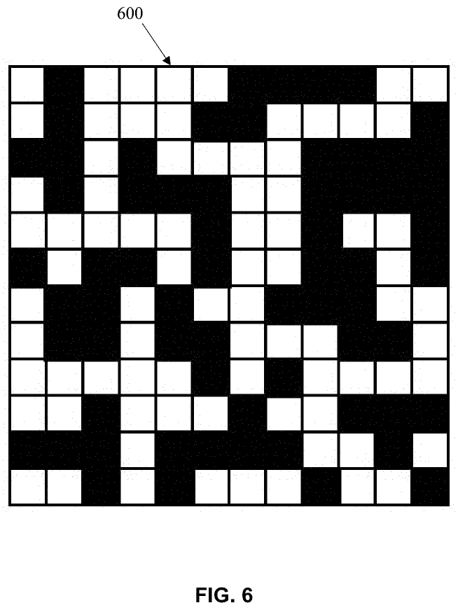

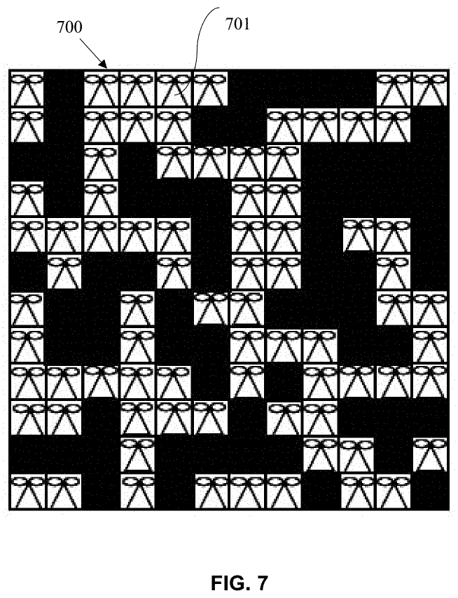

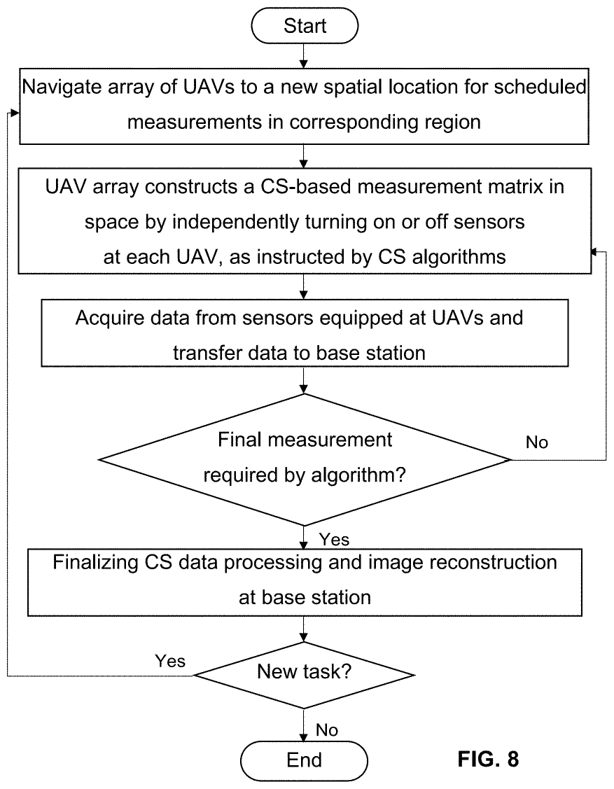

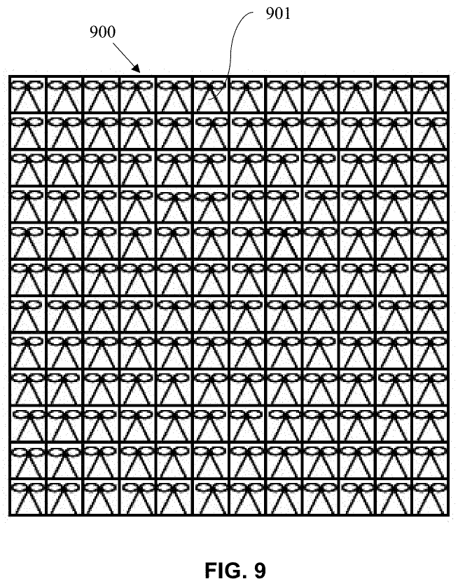

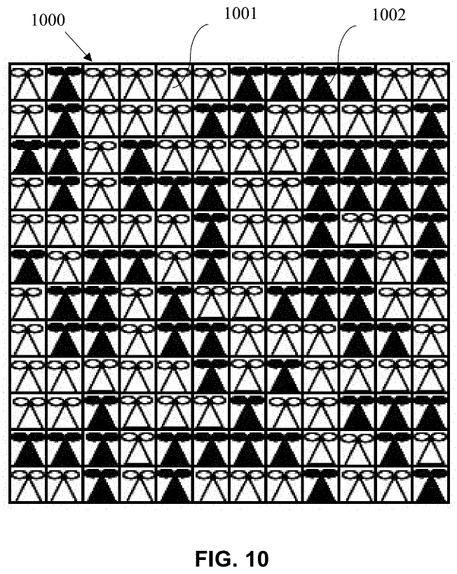

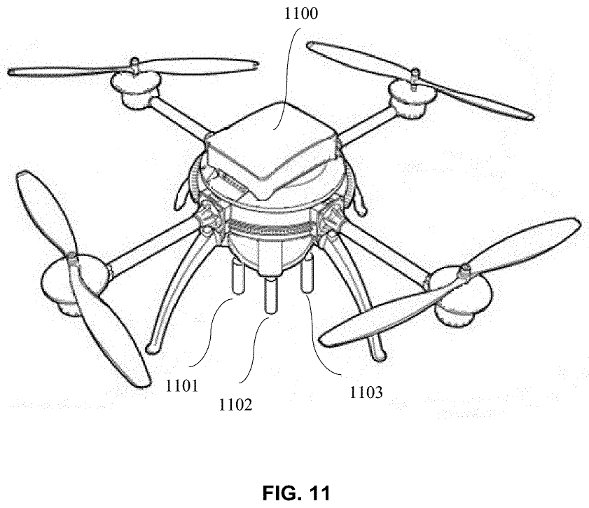

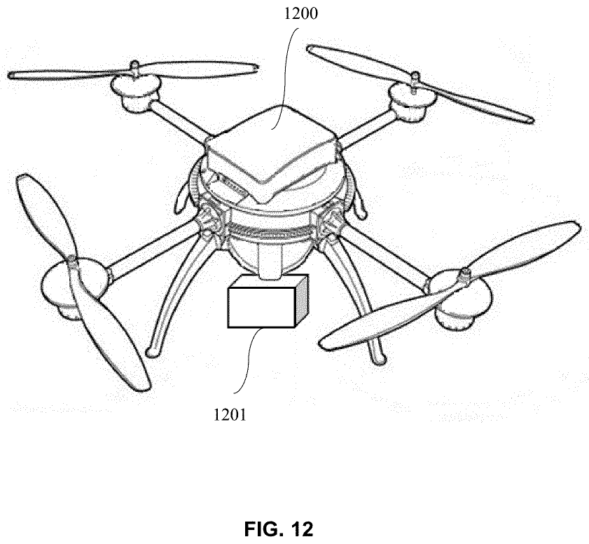

### DETAILED DESCRIPTION OF THE INVENTION

The terminology used herein is for the purpose of describing particular embodiments only but is not intended to be limiting of the invention.

The present disclosure proposed a novel remote sensing and imaging architecture to exploit a fleet of drones as a sensor array to work as a sparse sampling measurement matrix in compressed sensing algorithm.

The location of each drone, accurately controlled by GPS or other means to its coordinate in 3-D space, functions as a particular floating pixel in the CS matrix and the sensors mounted on it can be independently switched on or off, similar to the micro mirrors of DMD working in CS camera system. For each measurement in the sampling circle, the position of each drone in the fleet, and on or off status of the sensors in each drone are regulated by CS algorithm. Therefore, a flight pattern of a drone fleet is arranged to form a floating sparse matrix in the space, as required by the CS algorithm. The number of total measurements is determined by CS algorithm. Furthermore, through a higher level of computation, CS performs the signal recovery and reconstruction from the measurements coming from the output of the drone matrix, where the number of measurements is much fewer than the number of reconstructed pixels. Since the drone can fly in 3-D space, which add one dimension to the DMD camera system, the images can be reconstructed and presented in a multi-dimensional image with the CS data sets.

One or multiple sensors are mounted on each drone to provide sensor signals to the CS system. Any types of sensors, such as physical, or chemical, or optical, or any combination of type of the sensors mentioned above, can be employed to provide corresponding specific information to CS. For example, multiple hyperspectral sensors mounted on each drone will provide spectral image information across a multitude of spectral bands to enable CS algorithms to reconstruct hyperspectral images in different spectral bands.

The advantage of the invented architecture is that it is utilizing drones to work as floating sampling points of a sparse matrix in space to provide a means of mobilized, rapid, and economic multi-dimensional sensing and imaging methodology.

It is yet another advantage of the present invention, via multiple remote sensors mounted on the drone, to enable the drone to measure corresponding specific properties for a variety of substances and purposes simultaneously.

Finally, it is a further advantage of the present invention, due to variability and versatility of drone positioning adjustment, and the fact that CS sparse matrix contains fewer pixels resulting in much less drones needed in the fleet than actually measured pixels, to make invented architecture an ideal candidate for large-scale remote sensing, geodetic survey, rapidly deploying an area in urgent need, such as forestry fire, earthquake, explosion site, etc.

What is claimed is:

1. A remote sensing system includes at least a plurality of UAVs or drones fleet, mounted with plural airborne sensors, to construct a single or plural of sparse sampling measurement matrices in 3-D space for a compressed sensing algorithm.

2. During each compressed sensing sampling measurement, the position of each UAV or drone and the fleet pattern in said claim 1, are regulated according to measurement matrix design by said compressed sensing algorithm in claim 1.

3. Each UAV or drone in the fleet in said claim 1 works as a floating pixel in sparse sampling measurement matrix for said compressed sensing algorithm in claim 1.

4. The type of sensors in UAV or drone fleet in said claim 1 includes, but is not limited to, chemical sensors, physical sensors, thermal sensors, optical imaging sensors, spectral sensors, or any combination of the types of sensors mentioned above, for measuring corresponding signals.

5. Each remote sensor in UAV or drone fleet in said claim 1 can be independently switched on or off to output or to stop output signals.

6. The location control of each UAV or drone as set forth in claim 1 is provided by a global positioning system or other means.

7. A method of utilizing a plurality of UAVs or drones fleet to construct a sparse sampling measurement matrix in space for compressed sensing algorithms.
